(19) United States
(12) Patent Application Publication
(10) Pub. No.: US 2003/0099674 A1
(43) Pub. Date: May 29, 2003

(54) Lyophilized injectable formulations containing paclitaxel or other taxoid drugs

(76) Inventor: Andrew X. Chen, San Diego, CA (US)

Correspondence Address: Patrick D. Kelly, 11939 Manchester #403, St. Louis, MO, 63131, US

(21) Appl. No.: 10/218,083

(22) Filed: Aug. 12, 2002

Related U.S. Application Data
(60) Provisional application No. 60/311,302, filed on Aug. 11, 2001.

(51) Int. Cl. A61K 31/7048; A61K 31/337; A61K 9/00

(52) U.S. Cl. 424/400; 514/449; 514/27

(57) ABSTRACT

A stable and porous lyophilized cake or powder is disclosed, which contains paclitaxel or another water-insoluble taxoid drug. This preparation is created by dissolving a taxoid drug in oil and a surfactant, with each component selected to provide a final product that will be benign and gentle, compared to the harsher and more toxic carriers used in paclitaxel emulsions today. An alcohol can also be used during the drug mixing step, but it should be removed before subsequent processing. The oily solution is mixed with an aqueous solution containing an non-proteinous anti-adhesion agent with a collapse temperature preferably in a range of about −25° C. to about −35° C., such as sucrose. The mixture is processed to form an emulsion, with oil droplets averaging less than about 2 microns (and preferably less than 1 micron) in diameter. This emulsion is passed through a sterilization filter and loaded into vials, and is lyophilized to form a porous cake or powder which is stable and can be stored for long periods without refrigeration. The cake or powder can be reconstituted with water shortly before use, to form an injectable emulsion or suspension which does not contain harsh and potentially toxic solubilizing agents or surfactants, and which contains oil droplets with very small diameters.

### RELATED APPLICATION

This application claims priority under 35 USC 119 based on provisional application No. 60/311,302, filed Aug. 11, 2001.

### BACKGROUND OF THE INVENTION

This invention relates to pharmacology, and to improved injectable formulations containing insoluble taxoid drugs (such as paclitaxel, an important anti-cancer drug) that cannot be dissolved in water.

“Taxoid” drugs, as that term is used herein, includes taxine drugs, as that term is understood by those skilled in the art. This is a well-known and extensively studied class of compounds, several of which are widely used in cancer chemotherapy. The primary example is known as TAXOL™, the trade name given to an injectable formulation that contains an anti-cancer compound called paclitaxel. This compound was first isolated from the bark of a class of yew trees that grows in the northwest region of America. For more than a century, it has been known that the bark of yew trees is poisonous and even lethal, if eaten by livestock. Scientific interest in the underlying cause of its poisonous effects led to identification of a class of molecules called “taxines”. Testing and screening of those taxine compounds led scientists to a specific taxine compound that was given the name “paclitaxel”. Those screening tests indicated that paclitaxel is very potent in killing cancer cells in certain types of solid tumors, including breast cancer.

In nature, paclitaxel is found in significant quantities only in the bark of yew trees. Since the bark of trees cannot be harvested in large quantities without killing the trees, more research was done, and a way was discovered for chemically treating another taxoid compound which is found in the leaves of yew trees. Since leaves can be harvested in much larger quantities than bark, this led to a practical source of supply, and when ways were found for formulating paclitaxel into the injectable liquid called TAXOL, it became the largest-selling anti-cancer drug in the U.S. However, as discussed in more detail below, injection of TAXOL poses one of the most difficult and painful forms of cancer chemotherapy, largely due to the extremely hydrophobic and insoluble nature of paclitaxel and nearly all other taxine drugs that are of interest.

Because synthetic chemists and other researchers have developed numerous analogs and derivatives of taxine compounds, and since it is not always clear whether a particular analog or derivative falls within the formal definition of “taxine” compounds, the term “taxoids” is used to include taxine drugs as well as isomers, analogs, and derivatives of taxines that have molecular structures that are similar to taxines. In order to be included within the term “taxoid” as used herein, a taxine drug, or an isomer, analog, or derivative of a taxine drug, must be pharmacologically acceptable, and it must have a therapeutic medical utility in injectable formulations. Examples of water-insoluble taxoids that have been commercially used or reported in the scinetific literature as having been tested against cancer cells or solid tumors include paclitaxel; taxotere; taxane; spicatin; yunnanxol; taxane-2,13-dione,5β,9β,10β-trihydroxy-cyclic-9,10-acetal with acetone or acetate; taxane-2,13-dione,5β,9β,10β-trihydroxy-cyclic-9,10-acetal with acetone or acetate; taxane-2β,15β,9β,10β-tetrol-cyclic-9,10-acetal with acetone or acetate; N-debenzoyltaxol A; cephalomannine; cephalomannine-7-xyloside; 7-epi-10-deacetyl-cephalomannine; 10-deacetyl-cephalomannine; baccatin; baccatin diacetate; baccatin I through VI; 7-epi-baccatin III; baccatin A; 7-(4-azido-benzoyl)-baccatin III; O-acetylbaccatin IV; 7-(triethylsilyl)-baccatin III; 7,10-di-O-[(2,2,2-trichloroethoxy)-carbonyl]-baccatin III; 13-(2′,3′-dihydroxy-3′-phenylpropionyl)-baccatin III; baccatin III 13-O-acetate; taxol B; epitaxol; 10-deacetyl-7-epitaxol; 10-deacetyltaxol; 10-deacetyltaxol B or C; 7-xylosyl-10-deacetyltaxol; and 10-deacetyltaxol-7-xyloside. This list is not exhaustive; nevertheless, it is believed that all of the compounds listed above have been analyzed and discussed in the scientific literature, and may be of significant interest as drugs for anti-cancer or other medical purposes, especially now that a gentler and less-toxic method of administering them has been disclosed.

As used herein, the terms “soluble” or “insoluble” refer to the solubility of a taxoid drug in aqueous solutions (such as water, physiological saline, injectable dextrose solutions, etc). Water-soluble analogs or derivatives of taxoid drugs are of no interest herein, since this invention relates solely to drugs that cannot be dissolved at useful and therapeutic concentrations in aqueous carriers. The term “insoluble” as used herein can be used interchangeably with hydrophobic, lipophilic, oleophilic, and similar terms.

Because taxoid drugs tend to be highly toxic, and because injection of a taxoid in an intravenous injection or infusion allows much better control over blood-borne concentrations than other routes, such as oral ingestion, taxoid drugs are nearly always administered via injection of liquid solutions containing these drugs. Such liquids can also be referred to as “parenteral” formulations; this term can include any mode of pharmaceutical administration that does not go through the digestive tract, but it is most commonly used to refer to injections or infusions into blood vessels, and excludes trans-membrane delivery such as skin patches.

However, because of the very high levels of insolubility of taxoid drugs, intravenous injection (for convenience, this term includes both injections and infusions) poses serious problems and challenges for pharmaceutical scientists and physicians. Various methods for emulsifying, suspending, or encapsuling insoluble drugs in injectable formulations have been used for decades, but none of those approaches are fully satisfactory for taxoid drugs, and the “best available” formulations of TAXOL and other taxoid drugs pose serious problems, risks, and drawbacks. Such problems include, for example, severe pain at injection sites, high rates of allergic and/or immune reactions, serious and potentially permanent damage to blood vessels at or near the site of injection, and precipitation of insoluble drugs in blood vessels in ways that can lead to occlusion (blockage) of the affected vessels, which can lead to serious damage to the heart, brain, or other organs.

The limitations and shortcomings in the current state of the art for injecting insoluble taxoid drugs (and, indeed, for injecting any type of highly insoluble drug) can be better understood from a brief review of the most commonly used approaches to developing injectable formulations of insoluble drugs. The most widely used approaches known in the prior art include the following:

1. Use of organic solvents (such as ethanol, propylene glycol, polyethylene glycol, etc.), and mixtures of organic solvents with water. The problems that plague this approach include the following: (i) drugs prepared in this manner tend to precipitate when diluted with water, and precipitation poses especially severe problems for drugs that need to be infused over a span of minutes or hours, rather than injected in a single bolus within a few seconds; (ii) the stability of the drug molecules in the solvent poses a severe concern, because the drug molecules are being constantly exposed to, in effect, severe and constant jostling and even “bombardment” by the solvent molecules; and, (ii) serious localized irritation of tissue at or near the injection site is common, since tissue (including the interior surfaces of the walls of blood vessels) is not adapted or well-suited to being placed in close contact with high concentrations of organic solvents.

2. Use of emulsions with natural surfactants. Emulsions in this field of pharmacology refer to formulations in which tiny droplets of oily material (which hold the drug molecules) are intimately mixed with, and effectively suspended in, an aqueous carrier. Since the shelf life of a commercially useful drug compound typically needs to be measured in at least weeks or months, steps must be taken to prevent the tiny oil droplets from coalescing (aggregating) with each other, in a way that would form much larger droplets and eventually lead to separation of the mixture into a watery layer and an oily layer. To prevent the oily droplets from coalescing and separating, specialized molecules called “surfactants” (derived from the phrase “surface acting agents”) are used to stabilize emulsions. Several types of natural surfactants are known, including lecithin, certain types of fatty acid salts, and bile salts; however, when stored in liquid formulations, these are not adequately stable, and they are broken down (mainly by hydrolysis and oxidation) within periods of time that do not provide adequate and practical shelf lives for valuable drugs.

3. Use of emulsions with synthetic or processed surfactants. To extend the stability and shelf life of pharmaceutical emulsions, various types of synthetic or chemically-processed surfactants have been developed. Since their chemical names are usually long and complicated, they are commonly known by trade names, such as CREMOPHOR, POLYXAMER, and TWEEN. They are widely used; as one example, CREMOPHOR is the emulsifying agent used in nearly all TAXOL and other taxoid formulations that are being injected into cancer patients today. However, synthetic and chemically processed surfactants tend to cause painful local reactions, and most patients must be anesthetized (or at least sedated) before they can be injected with these compounds.

In addition, since synthetic or processed surfactants act in a manner comparable to detergents, they inflict their own forms of systemic toxicity on cells and tissue; therefore, the amount of a synthetic or processed surfactant which can be injected into a patient is very limited. This type of systemic toxicity poses serious problems, and severely aggravates the dilemmas that arise when a gravely ill patient suffering from potentially terminal cancer must be treated. In such cases, a large dosage of the anti-cancer drug must be used in order to kill aggressively-growing cancer cells, but just as clearly, a patient who is already weakened by cancer will be hammered even harder, if a large quantity of a nasty detergent-like material with toxic side effects of its own is injected directly into his or her veins and arteries.

In addition, when surfactants are used to stabilize emulsions, other problems must also be taken into account. Such problems include: (i) a certain drug may have only low solubility in the types of non-toxic oily materials (such as vegetable oils) that can be used safely in injectable emulsions; (ii) the volume of oil that can be stably suspended in an emulsion is commonly only about 20% or less, by volume; this requires large volumes of emulsion (and correspondingly large quantities of surfactant) to be injected into a patient; (iii) a certain drug may not be sufficiently stable to give it practical shelf life, if it is fully dissolved in an oily carrier material; (iv) any problems with stability in an oily carrier often become worse, if the drug molecules also will be intimately contacted by a detergent-like surfactant.

4. Use of suspended particulates. Some insoluble drugs can be prepared as very tiny particulates (with average diameters of a few microns, or less), which are suspended in an aqueous liquid. Problems that limit and plague these types of suspensions include: (i) the difficulties of manufacturing suspensions with reliable and consistent particle sizes; (ii) problems with hydrolysis and other chemical degradation of the particulates suspended in the water; (iii) problems with the particulates settling to the bottom or floating to the top of the aqueous liquid, in a way which typically requires vigorous agitation and resuspension immediately before use.

5. Use of liposomes. Methods have been developed for using certain types of lipid molecules which, when mixed vigorously in water, will spontaneously arrange themselves into tiny spheres formed by bilayer membranes. If the manufacturing operation is carried out in a certain manner, the spheres can be made to enclose hydrophobic molecules that are trying to minimize their area of surface contact with the surrounding water molecules. Although this approach is useful for some drugs and promising for others, it suffers from various problems, including: (i) low levels of incorporation efficiency, leading to wastage or expensive recovery efforts for the non-incorporated drug molecules, and higher costs for the final drug products, and (ii) difficulties in quality control and in preparing reliable final products with liposome sizes in a consistent and narrow range.

6. Use of complexing agents. A few types of drugs can be rendered more soluble in aqueous solutions, by the use of specialized complexing agents, such as cyclodextrins or niacinamide. However, this is not really a generally useful approach; only certain specific drugs can be properly complexed in a way that renders the drug quasi-soluble in water. In addition, complexing tends to be a low-efficiency process that requires large quantities of complexing agent, which increases the costs of the final product, and complexed drugs of this type tend to suffer from precipitation problems.

All of these approaches, and the problems that hinder them and limit their use, are described in various textbooks and review articles known to those who work in this field. In addition, in evaluating any of these types of approaches for potential use with a specific drug, it must be borne in mind that three essential goals must be met. Those three goals are (1) drug safety; (2) tissue compatibility; and, (3) stability and shelf life. Any solubilizing, suspending, emulsifying, or similar agent or method must lead to a formulation that: (i) must have sufficiently low systemic toxicity to allow its therapeutic use, in patients who may be gravely ill and severely weakened; (ii) is adequately compatible, even at high concentrations, with tissues at or near the injection site; and, (iii) are sufficiently stable, during shipping, storage, and use, to be commercially practicable. Thus, a solubilization technique simply will not be useful, no matter how promising it might seem, if these key criteria cannot be satisfied.

Unfortunately, almost every solubilizing, suspending, emulsifying, or similar technique currently used by formulation scientists suffers from inherent shortcomings. The real-world results and effects of these problems and shortcomings can be seen by evaluating products that are actually being sold and used as of the date this is being written, and considering what real patients must go through when being treated by these drugs. TAXOL (injectable paclitaxel) offers a prime example, since it is one of the largest selling drugs anywhere in the world. Despite the fact that hundreds of millions of dollars have been spent on research involving TAXOL, injection of TAXOL remains one of the most difficult and painful forms of cancer chemotherapy. In order to solubilize paclitaxel in an aqueous injectable liquid, ethanol is used as a solvent, and polyoxyethylated castor oil (widely known by the trademark CREMOPHOR) is used as a surfactant. Both of these agents (ethanol and CREMOPHOR) are required to keep the pacilitaxel in a suitable dissolved and/or suspended state in the liquid.

However, while ethanol and CREMOPHOR help create a stable and usable formulation, their combination is toxic and painful. Ethanol (ethyl alcohol) has its own forms of systemic toxicity, when ingested orally; when injected directly into a vein, it becomes substantially more toxic and dangerous. CREMOPHOR is also toxic and has serious adverse side effects, in large numbers of patients; when injected intravenously, by itself, it can produce hypersensitivity reactions, including anaphylactoid reaction, leading to potentially fatal results. These undesirable adverse effects are commonly encountered in clinical practice, and in at least one case, a cancer patient died as a direct response to a TAXOL injection.

In an effort to prevent and minimize these adverse effects and risks, all cancer patients receiving TAXOL are routinely pre-medicated, to prevent or reduce severe hypersensitivity reactions, and TAXOL treatment often requires or strongly suggests hospitalization, as a standard precautionary measure for cancer patients. The requirements for pre-medication and hospitalization contribute substantially to the high cost of TAXOL treatment.

Another example of a flawed and limited solubilization technique involves diazepam (more widely known by the trademark VALIUM). Oral diazepam is an important and widely used drug for managing anxiety disorders. Injectable diazepam is also used, much less widely, for several classes of patients, including patients suffering from seizures or severe anxiety disorders, and patients undergoing withdrawal from alcohol or drug dependence. It is also used in patients being prepared for surgery, since it can help control and reduce “emergence psychosis” and other adverse reactions to certain types of surgical anesthetics. However, injectable formulations of diazepam pose serious risks and problems. Like paclitaxel, diazepam is insoluble, and requires a combination of propylene glycol and ethanol for solubilization. It is known to cause high incidence rates of venous thrombosis, phlebitis, local irritation, and vascular impairment, and the packaging warnings clearly state that it must not be injected into a small vein, or into an artery. At least some of its problems are believed to be caused or aggravated by drug precipitation in blood vessels, after the drug solution has been injected into a vein.

In practice, injectable emulsions offer a very good way to deliver certain types of nutrients; however, their actual use in delivering injectable drugs is very limited. Because several types of major nutrients are oily in nature, oil-in-water nutrient emulsions have been widely used; examples include Intralipid (marketed by Pharmacia), Lipofundin (Braun), Liposyn (Abbott), and Travamulsion (Baxter). However, examples of intravenously injectable emulsions containing, active drugs, as compared to nutrients, are rare. DIPRIVAN (an injectable emulsion containing propofol, a surgical anesthetic sold by AstraZeneca) and DIAZEMULS (a diazepam emulsion, marketed in Europe by Pharmacia) are the only two examples known to the Applicants herein, in the U.S. and Europe.

When an injectable emulsion which contains an active drug in the oil phase (i.e., in tiny oil droplets that are suspended in a continuous water phase) is used, three distinct stability concerns must be confronted. Those are: (i) the stability of the drug in the oil phase, especially if the drug molecules will contact surfactant molecules; (ii) the stability of the oily droplets in the water phase, as indicated by average diameters of the droplets, which often tend to coalesce and grow over weeks or months of storage; and, (iii) the stability of the surfactants and other excipients, which must remain stable in order to keep the tiny droplets of oil properly suspended in the water phase of the emulsion.

For some drugs, reduced levels of hydrolytic degradation can be achieved by incorporation of drugs into the oily droplets in an emulsion. However, for other drugs, the presence of the aqueous phase causes serious problems with hydrolysis. For example, prostaglandin E1, an important drug for the treatment of arterial occlusive disease, might appear to be an ideal candidate for delivery in an emulsion; however, it tends to have low storage stability and low shelf life in emulsions, owing to hydrolytic degradation.

The physical stability of the oily droplets in emulsions also poses serious challenges and problems. The two major classes of physical changes that tend to occur over weeks or months of storage are: (i) creaming, in which the lipid droplets (which are almost always less dense than water) tend to slowly rise to the top of the aqueous phase, where they gather in an oily layer on top of the aqueous layer; and, (ii) processes that can be called aggregation, flocculation, or coalescence, in which the oily droplets irreversibly merge together to form larger droplets, or cluster together to form clumps of droplets, which may still have distinct membranes but which will not perform in a satisfactory manner following injection. These types of gradual changes inevitably lead to an increase in droplet size.

Even if an increase in droplet size does not-lead to a “broken” emulsion, any increase in droplet size poses a serious problem for an injectable formulation. For intravenous injection, the oily droplets in an emulsion should always be less than about 5 microns; preferably, they should be less than about one or two microns, to minimize risks of embolism (i.e., blockage which is caused by a discrete particle, as distinct from a gradual buildup of fatty deposits on the interior walls) in the capillaries. Because of the risk of embolism, any increase in droplet size in an emulsion, even if it occurs only slowly over a span of weeks or months, is a critical stability and safety issue.

In addition, gradual degradation of the surfactant used in an emulsion poses yet another concern regarding stability and safety of the emulsion. This is especially true when natural surfactants (such as lecithin or phospholipids) are used. Natural surfactants such as lecithin (which contains phosphatidylcholine as a major component) are susceptible to oxidation and hydrolysis, resulting in degradation products (such as lyso-phosphatidylcholine) that can produce irritation, and that do not function effectively as surfactants.

Because of various factors, emulsions have been regarded as holding great promise for creating injectable formulations of insoluble drugs. However, because of the problems that plague the chemical and physical stability of emulsions, injectable emulsions simply have not become practical or widespread in the drug industry, and only a very small number of drugs have ever been commercialized in injectable emulsion formulations. As noted above, there are only two injectable emulsions in widespread current use; those are DIPRIVAN, which usually must be accompanied by lidocaine treatment, to minimize localized pain at the site of injection, and DIAZEMULS, which is used mainly for treating people suffering from severe convulsions, and for treating alcoholics and addicts who are suffering intense physical discomfort due to withdrawal from alcohol or opiates.

Because of the problems that plague liquified emulsions, a number of researchers have attempted to create “freeze-dried” (lyophilized) emulsions. In these efforts, the process of lyophilization subjects a liquified mixture having an aqueous phase to a very cold temperature, which causes the water to freeze into ice. The ice is then removed by a process called sublimation; during this process, the frozen mixture is subjected to an intense vacuum. Since ice has a low but significant level of “vapor pressure”, the vacuum gradually pulls the molecules of water out of and away from the ice, and removes them, in a manner comparable to the way a “frost-free” freezer keeps ice from accumulating, by using a high level of air flow inside the freezer. Over the course of one to two days, the intense vacuum inside a lyophilization chamber can thereby remove the water from an emulsion, leaving behind just the oily droplets containing the insoluble drug.

Various scientists have indeed attempted to formulate drug-containing lyophilized emulsions for intravenous injection; however, none of those efforts have yet succeeded on a noteworthy scale. For example, several such efforts have led to the use of excipients that are not considered as safe, or are not accepted by regulatory agencies as injectable excipients. For example, U.S. Pat. No. 5,750,142 (Friedman et al 1998) describes lyophilized emulsions which use medium-chain triglycerides (MCT) as the oil phase, and which also contain a primary surfactant, a co-surfactant, and a bulking agent. The surfactants disclosed in that patent included ethoxylated and/or propyoxylated alcohol or esters, such as POLYXAMER, POLYSORBATE 80 and 20, POLYOXYL 40 sterarate. However, these synthetic surfactants are known to be associated with hypersensitivity reactions (e.g., Tyson et al), and the use of a POLYSORBATE-containing injectable drug is likely to require pre-medication with an anti-histamine drug or dexamethasone (e.g., see the warnings in the Physicians Desk Reference concerning TAXOTERE). In addition, protein hydrolysate or PVP, which were disclosed as the preferred bulking agents in U.S. Pat. No. 5,750,142, very probably would not be regarded as safe excipients for an injectable drug, if present at the concentrations required for the lyophilized emulsions described in that patent.

Two other US patents, U.S. Pat. No. 5,635,491 (Seki et al 1997) and U.S. Pat. No. 5,977,172 (Yoshikawa et al, 1999) describe other attempts to create lyophilized emulsions. However, these patents specifically state that maltose was the only useful freeze-drying aid for those lyophilized emulsions. The safety of maltose in injectable formulations is unknown, and to the best of the Applicants' knowledge, the FDA has never approved any intravenously injectable drug which contains maltose.

U.S. Pat. No. 5,882,684 (Schutz et al 1999) describes yet another effort to create a lyophilized emulsion. The formulations described in this patent employed acetylated monoglyceride as the oil phase, and glycerol polyethylene glycol ricinoleate or polyoxyethylene 660 12 hydroxystearate as the emulsifier. However, neither of those two classes of agents are regarded as safe excipients for injectable formulations; instead, they are more commonly used in the food industry.

Krishna et al 1999 reported an attempt to lyophilize emulsion formulations containing soybean oil, lecithin, and sorbitan monolaurate (also known as SPAN 20) as emulsifiers, and a polyhydroxy alcohol as the bulking agent. However, after the lyophilization process has been completed and the dried emulsion has to be “reconstituted” back into liquid form by mixing it with water again, the particle size of the reconstituted emulsion was found to increase substantially. Most of the lyophilized particles formed from the frozen oily droplets were found to have poor homogeneity, or had collapsed. The only product which reportedly had a satisfactory level of uniformity contained 30% glycerol, and existed as a non-aqueous liquid, rather than as a dried solid; therefore, it did not provide a dried emulsion at all.

Accordingly, it appears that all known previous efforts at creating freeze-dried emulsions have fallen short of the needs and constraints that will apply to a useful and practical freeze-dried emulsion that can be reconstituted into a safe and effective liquid form, shortly before injection into a human patient.

In addition, substantial prior art has been published which describes various attempts to develop improved formulations containing paclitaxel and other taxoid drugs. Recent US patents that describe such efforts include the following:

U.S. Pat. No. 6,365,191 (Burman et al 2002), U.S. Pat. No. 6,355,273 (Carli et al 2002), U.S. Pat. No. 6,338,859 (Leroux et al 2002), U.S. Pat. No. 6,322,805 (Kim et al 2002), and U.S. Pat. No. 6,284,746 (Szente et al 2001), all of which describe the use of polymers to create very tiny microspheres that assertedly will release a water-insoluble taxoid drug after injection.

U.S. Pat. No. 6,046,230 (Chung et al 2000), U.S. Pat. No. 6,107,333 (Andersson 2000), U.S. Pat. No. 6,040,330 (Hausheer et al 2000), U.S. Pat. No. 6,017,948 (Rubinfeld et al 2000), U.S. Pat. No. 5,965,603 (Johnson et al 1999), and U.S. Pat. No. 5,922,754 (Burchett et al 1999) describe the use of various other synthetic agents (such as dimethylacetamide, povidone, various pyrrolidones, saccharide fatty acid esters, polyglycol esters of hydroxystearic acid, etc.) to form taxoid suspensions.

U.S. Pat. No. 5,407,683 (Shively et al 1995) and PCT application WO 02/26208 (Constantinides et al) disclose efforts to provide emulsions containing taxoid drugs that are dissolved in specialized types of oils, such as squalene or squalane oil (which can be derived from certain types of marine organisms), or Vitamin E (a very oily hydrophobic compound, also known as alpha-tocopherol, which can serve as both a carrier vehicle and an anti-oxidant).

U.S. Pat. No. 6,348,215 (Straubinger et al 2002), U.S. Pat. No. 6,296,870 (Needham 2001), U.S. Pat. No. 6,143,321 (Needham et al 2000), U.S. Pat. No. 6,146,659 (Rahman 2000), and U.S. Pat. No. 5,653,998 (Hamann et al 1997), all of which describe processes for enclosing a taxoid drug within liposomes.

U.S. Pat. No. 6,319,943 (Joshi et al 2001), U.S. Pat. No. 6,294,192 (Patel et al 2001), and U.S. Pat. No. 6,284,268 (Mishra et al 2001), all of which attempt to create taxoid formulations that can be ingested orally, rather than having to be injected.

U.S. Pat. No. 6,251,428 (Yoo 2001), which describes the use of bile acids and either dextran or liquid glucose, to create clear solutions that assertedly will not form precipitates over certain pH ranges; and,

U.S. Pat. No. 6,140,359 (Carver et al 2000), U.S. Pat. No. 6,306,894 (Carter et al 2001), and U.S. Pat. No. 6,071,952 (Owens et al 2000), U.S. Pat. No. 5,925,776 (Nikolayev et al 1999), and U.S. Pat. No. 5,681,846 (Trissel 1997) all describe efforts to create improved taxol emulsions that still contain Cremophore, but in an improved pH-modified form.

In addition to the foregoing, several efforts have been made to create improved liquid emulsions and/or micelle preparations that contain taxoid drugs. One such efforts is described in U.S. Pat. No. 5,616,330 (Kaufman et al 1997), which describes a liquid emulsion formed with long-chain triglycerides, using an alcohol such as isopropanol during a processing step and then removing the alcohol before creating the emulsion. Although that process is similar in some respects to certain steps used in the preparative method disclosed herein, the product created by Kaufman et al is stored as a liquid emulsion, and therefore is substantially different from a lyophilized cake or powder as described herein.

One object of this invention is to disclose methods and reagents for preparing long-lasting and stable freeze-dried (lyophilized) emulsions, which contain highly insoluble drugs such as paclitaxel or other taxoids, which do not require refrigeration, and which can be reconstituted by mixing with water shortly before use, to provide a safe and stable emulsion with very small droplet sizes, for injection into humans.

Another object of this invention is to disclose improved methods and reagents for preparing lyophilized emulsions, which can subsequently be used safely and effectively as injectable emulsions containing drugs that cannot be dissolved in water.

Another object of this invention is to disclose improved agents for coating the frozen oily particulates that are generated when an drug-containing emulsion is lyophilized, to ensure that the frozen oily particulates will not aggregate or suffer from other problems during storage or reconstitution.

These and other objects of the invention will become more apparent through the following summary, drawings, and description.

### SUMMARY OF THE INVENTION

A stable and porous lyophilized cake or powder is disclosed, which contains paclitaxel or another water-insoluble taxoid drug. This preparation is created by dissolving a taxoid drug in oil and a surfactant, with each component selected to provide a, final product that will be benign and gentle, compared to the harsher and more toxic carriers used in paclitaxel formulations today. An alcohol can also be used during the drug mixing step, but it should be removed before subsequent processing. The oily solution is mixed with an aqueous solution containing an non-proteinous anti-adhesion agent with a collapse temperature preferably in a range of about −25° C. to about −35° C., such as sucrose. The mixture is processed to form an emulsion, with oil droplets averaging less than about 2 microns (and preferably less than 1 micron) in diameter. This emulsion is passed through a sterilization filter and loaded into vials, and is lyophilized to form a porous cake or powder which is stable and can be stored for long periods without refrigeration. The cake or powder can be reconstituted with water shortly before use, to form an injectable emulsion or suspension which does not contain harsh and potentially toxic solubilizing agents or surfactants, and which contains oil droplets with very small diameters.

### BRIEF DESCRIPTION OF THE DRAWINGS

FIG. 1 is a flowchart which summarizes the major steps in a preferred method of the invention.

FIG. 2 is a two-chamber vial, showing a lyophilized taxoid drug in the lower chamber, separated from water in the upper chamber by a rubber plug, which can be dislodged by depressing a plunger cap, to allow mixing of the taxol with the water, inside the vial.

### DETAILED DESCRIPTION

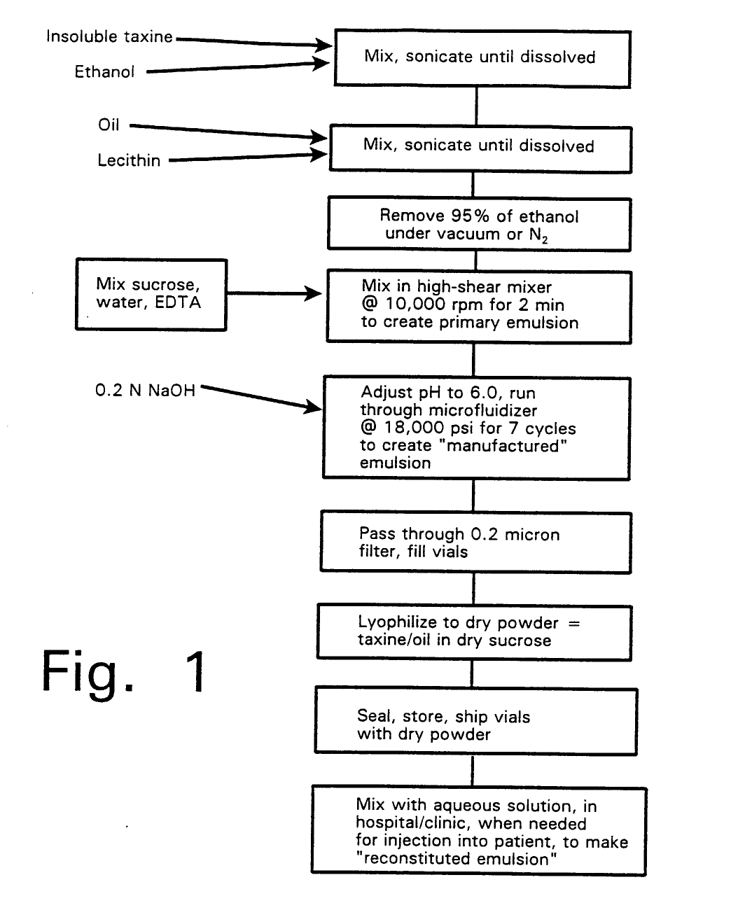

As summarized above and depicted in the flowchart in FIG. 1, the manufacturing process of this invention creates an emulsion containing a water-insoluble taxoid drug (such as paclitaxel), dissolved within droplets of an oily carrier compound suspended in an aqueous liquid. This emulsion, which also contains an anti-adhesion compound such as sucrose, is loaded into vials and lyophilized (i.e., frozen, at a temperature which is below the “collapse temperature” of the anti-adhesion agent, and then subjected to an intense vacuum to remove the ice), to convert it into a dried cake or powder. The dried cake or powder is stable and has a long shelf life (expected to be well over a year) when stored in a sealed vial, even when stored at room temperature and not refrigerated. When a dosage of the taxoid drug is needed for a patient in a hospital or clinic, the cake or powder is reconstituted into an emulsion or suspension, by mixing it with sterile water or other injectable aqueous liquid, such as a buffered saline or dextrose solution. The cake or powder is porous, and the anti-adhesion agent will dissolve readily in water; therefore, the lyophilized preparation can be reconstituted into an emulsion using mild agitation (as used herein, this refers to mixing by hand, such as by simply shaking a vial), and will not require machine processing.

All steps used in preparing the emulsion can be carried out using conventional equipment known to those skilled in the art, such as a sonicator to help dissolve the taxoid drug in alcohol and subsequently in oil, a high-shear mixer to convert the water-oil mixture into a primary emulsion, and a microfluidizer to convert the primary emulsion into a sub-micron emulsion that is ready to be lyophilized. A preferred series of steps for carrying out this part of the manufacturing process is shown in FIG. 1; however, as will be recognized by those skilled in the art, various modifications to the sequence or substance of any of those steps can be made, so long as the final result is a taxoid-containing emulsion with sufficiently small droplet sizes (which should be less than about 2 microns, and which preferably should be less than 1 micron, in average diameters) which is ready to be lyophilized.

Lyophilization also can be carried out using standard equipment. To eliminate the need for post-lyophilization grinding or other processing which might jeopardize sterility, the lyophilization step can be carried out on emulsion which has been pre-loaded into the vials that will hold it and keep it sterile during shipping, storage, and handling. Before or during the vial loading stage, the emulsion can be passed through a sub-micron sterilizing filter which has a sufficiently small pore size to remove any bacteria or viruses.

As used herein, the term “vial” refers to any stiff-walled container that is used to hold a lyophilized drug. Nearly all pharmaceutical vials are made of clear glass, which allows several advantages, including visual inspection of the enclosed drug (to ensure that it is still in a clean, non-caramelized, non-collapsed form, when it is ready for use) and of the container itself (to ensure that it does not have a hairline crack in one of the walls, which could jeopardize or destroy sterility of the enclosed drug). Various types of pharmaceutical vials are known. Single-chamber vials can be sealed with rubber or plastic plugs that will allow a hypodermic needle to be pushed through the rubber seal. Alternately, a single-chamber vial can be made of a brittle and easily breakable material, inside a sealed bag that can contain an aqueous solution (such as physiological saline or a dextrose solution, in an intravenous infusion bag); if this type of vial is broken, it will release its contents into the still-sealed bag, for mixing.

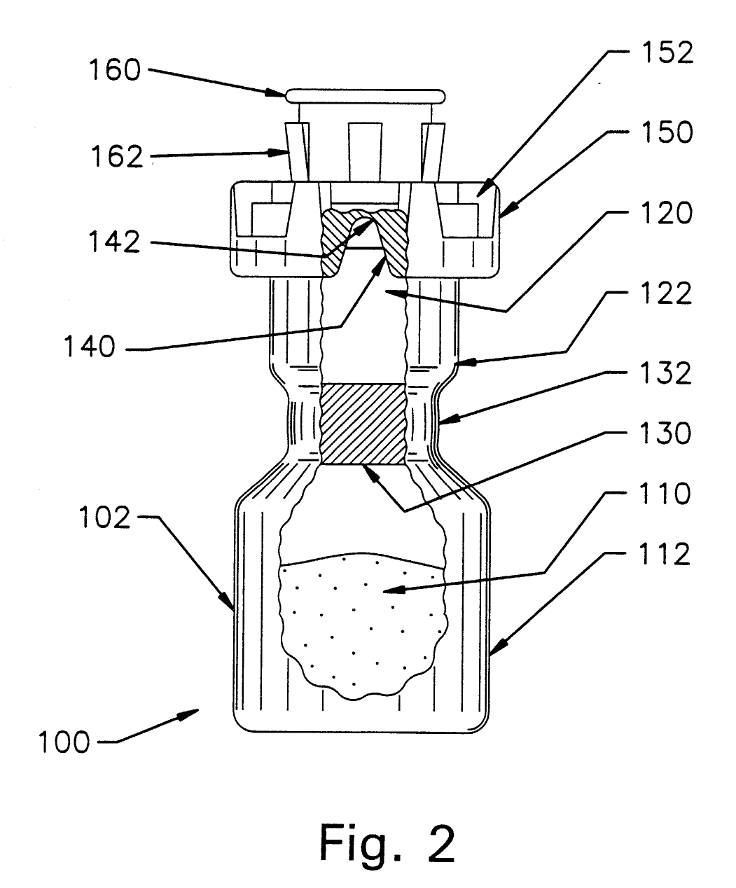

A conventional two-chamber vial assembly 100 is shown in FIG. 2; this type of vial device is shown in various US patents, including U.S. Pat. No. 4,781,354 (Potts 1981). This vial assembly comprises an outer wall 102 which is roughly cylindrical with a flat bottom, and which defines and enclosed a lower chamber 112 (which must be filled first, during the manufacturing process) and an upper chamber 122, separated from each other by a narrower constriction band 132.

This vial structure is conventional and well-known, and the point of novelty of the vial shown in FIG. 2 is that it contains a lyophilized taxoid cake or powder 110, as disclosed herein, in lower chamber 112. The vial also contains sterile water 120 (preferably in a solution intended for injection that also contains other conventional ingredients such as dextrose, Ringer's lactate, etc.), in upper chamber 122.

The two chambers 112 and 122 are separated from each other by a water-tight partition or “septum” 130, which is a non-permeable disk made of an inert flexible material such as butyl or silicone rubber. Constriction band 132 holds septum 130 in place until the vial of taxoid drug is needed.

A second non-rigid plug 140, usually made of butyl rubber, is mounted in the neck of the vial, and is secured to the vial by means of a metallic cap 150. This soft, flexible plug 140 allows a sharp tip of a hypodermic or tubing needle to be inserted into upper chamber 122, through a relatively thin upper wall portion 142 of the plug 140. This allows removal of a reconstituted liquid from the vial, so that the liquid can be loaded into a hypodermic syringe or infusion bag, for injection into a patient.

The metallic cap 150 interacts with plunger 160, allowing the plunger to be forced down, through an orifice which occupies the center of the cap 150. Outward-extending locking ears 162 in the sides of plunger 160 interact with accommodating slots 152 in the metallic cap, to lock the plunger in position once it has been pushed down into the cap.

When a patient is ready and the taxoid drug is needed for injection, plunger 160 is depressed. This pushes septum 130 out of position in the constriction band 132, aided by two factors: (a) the inert gas (usually nitrogen or argon) that fills the top of lower chamber 112 is compressible, and allows downward motion of the septum 130 under pressure; and (2) the aqueous liquid which fills the upper chamber is non-compressible, and causes the full force placed on plunger 160 to be pressed against the movable septum 130.

As soon as the septum 130 falls into the lower chamber 112, the dry lyophilized taxoid 110 comes into contact with the aqueous solution 120, and the two are mixed together thoroughly, by shaking the vial. The septum can bounce around inside the lower chamber 112 during this shaking process, and acts as a mechanical agitator to promote full mixing, and to help rapidly break apart the cake, if the taxoid 110 is in a solidified cake form rather than a powder.

These and other types of vials (and related and other methods of containment and use) are well-known to those skilled in the art. The type of vial or other containment system used to ship, store, or handle the cake or powder disclosed herein is not critical to this invention; any suitable system can be used, provided that it allows the cake or powder containing the taxoid drug to be reconstituted into an emulsion, in a safe and sterile manner.

In the preparations created to date, the concentration of taxoid in the pre-lyophilized emulsion was in the range of about 0.1% to about 0.5%, by weight. When the lyophilized cake or powder is mixed with water (presumably in a hospital or clinic) to reconstitute an injectable emulsion shortly before use, the drug concentration in the reconstituted emulsion can be controlled easily, merely by adding more or less water.

For convenience, the reconstituted mixture is referred to herein as an emulsion, although it may be either an emulsion or a suspension, depending on how much water is added to the cake or powder, and how vigorously the water-plus-powder mixture is mixed together before injection. When referring to reconstitution of the lyophilized drug, the term “water” includes any aqueous solution suited for injection or infusion; this includes, for example, a buffered saline solution, or a solution containing a sugar such as dextrose (the D-isomer of glucose).

For purposes of description, it is presumed that sufficient water will be added to the cake or powder to reconstitute an emulsion that will have roughly the same volume and concentrations that were present in the manufactured emulsion, before lyophilization. Since concentrations of about 2 to 3 milligrams of paclitaxel per milliliter of fluid are in a generally preferred range for cancer treatment, it is presumed that similar concentrations of paclitaxel in the pre-lyophilized emulsion are also desirable. However, substantially higher taxoid concentrations can be achieved, using the methods and compounds disclosed herein.

Oil concentrations in preparations created to date generally ranged from about 2% to about 30% of the manufactured emulsion weight, and surfactant quantities generally ranged from about 1% to 15% of the emulsion weight. The anti-adhesion compound (such as sucrose) was usually present at a range of about 5% to 30% by weight.

Every carrier component (as used herein, this term includes the oily material, the surfactant, the anti-adhesion agent, and any anti-oxidants, stabilizers, or other components other than a taxoid drug or other active drug) which is selected for inclusion in the lyophilized product should be benign and well-suited for injection, even in gravely ill patients. As used herein, terms such as “benign” are interpreted by using CREMOPHOR, and CREMOPHOR-containing formulations, as a benchmark for comparison. A surfactant or other carrier mixture is regarded as “benign” if it is significantly less irritating and troublesome, to typical and average patients, than the standard CREMOPHOR-containing TAXOL formulations being sold and used today. In general, benign carrier compounds include compounds that can be administered intravenously, without requiring advance sedation of a patient to avoid a painful or hypersensitive reaction at the injection site. Suitable oils, surfactants, and anti-adhesion agents which fall within this category are discussed in more detail below.

If desired, an alcohol component can be used to help create the initial drug-in-oil mixture, to increase the solubility of the taxoid compound in the chosen type of oily carrier. The great majority of this alcohol will be removed in a vacuum-drying step, before the taxoid-in-oil mixture is suspended in water to form the manufactured emulsion. Accordingly, the selected alcohol does not need to be nontoxic, and can be selected based on its solubility-enhancing traits. Nevertheless, highly toxic alcohols (such as methanol) generally should be avoided, since they may raise concerns over physiological effects if trace amounts remain in the cake or powder. Ethanol has provided good results in tests done to date.

Preferably, another agent (referred to herein as an “anti-adhesion” agent) also should be included in the emulsion, by dissolving it in the water before the water is mixed with the taxoid-in-oil mixture. This type of agent can also be called a bulking agent, a filler or filler agent, a matrix-forming agent, a particle-coating agent, or similar terms. The term “cryo-protectant” can also be applied to this compound, since one of its important functions is to prevent collapse of the cake or powder during the lyophilization process; however, some researchers may use and define the term “cryo-protectant” in different ways, so that term should be used with caution. Anti-adhesion agents are discussed in more detail below.

Suitable conditions for (i) the vacuum-drying step, which removes the alcohol from the oil-taxoid mixture before water is added to create the emulsion, and (ii) the lyophilization step, which removes the water form the emulsion, can be developed for any combination of specific selected constituents, at any preferred concentrations, using routine experimentation, using methods and equipment well-known to those who work with lyophilized drugs. The examples set forth below describe various combinations of time, temperature, and vacuum that have performed well in test batches prepared to date. It should be noted that, to prevent the collapse of frozen taxoid emulsions as described herein, temperatures during lyophilization should be kept below about −25° C., preferably below about −28° C., and even more preferably below about −32° C.

Another advantage of this invention that deserves note is that a lyophilized cake or powder will tend to suffer fewer losses and deterioration during storage than a liquid emulsion. This is due to the fact that in a liquid emulsion, the oily droplets have a substantially greater tendendy to cling and adhere not just to each other, but to any solid surface (glass, plastic, etc.) inside the vial, during storage. The products disclosed herein do not appear to suffer from such problems.

Carrier Compounds

The methods of this invention can be adapted for use with various types of taxoid drugs, various types of oils, various types of surfactants, and various types of anti-adhesion compounds. Any such compounds (including those listed below) can be evaluated for use as disclosed herein, using no more than routine experimentation, to determine whether they can be used to create a stable and satisfactory lyophilized cake or powder containing a taxoid drug.

A. Oils

The term “oil” is used herein in a general sense to identify hydrocarbon, carbohydrate, or similar organic compounds that are liquid at room or physiological temperature, and that are pharmacologically acceptable in injectable formulations. This class includes a variety of known vegetable oils, animal fats, and synthetic oils, as well as various liquids that are obtained by chemical treatment of such oils and fats.

The oil may be any of a number of oils commonly found in plants or animals, or any of various non-toxic synthetic oils; it may also be a mixture of any of these types of oil. Suitable candidates, any of which can be evaluated for use as described herein, include sesame oil, soybean oil, safflower oil, corn oil, cottonseed oil, peanut oil, palm oil, etc. Because paclitaxel was found to be somewhat more soluble in sesame oil than in the other oils that have been tested to date, sesame oil was used in various tests disclosed in the examples.

It is generally presumed that glycerides formed by reacting medium-chain fatty acids with glycerol (these are the predominant forms of animal fats and vegetable oils) are generally preferred; this includes triglycerides, diglycerides, and monoglycerides formed from medium-chain fatty acids. Alternately, oils formed by creating ester bonds between fatty acids and various alcohols other than glycerol can also be evaluated for such use, if desired.

Within the “vegetable oil” category, oils are derived mainly from seeds or nuts, and include corn oil, safflower oil, soybean oil, cottonseed oil, peanut oil, olive oil, rapeseed oil, coconut oil, palm oil, etc. A typical vegetable oil is a long-chain “triglyceride” molecule, formed when three fatty acids (usually 16 to 18 carbons in length, with unsaturated bonds in varying numbers and locations, depending on the source of the oil) form bonds with the three hydroxy groups on glycerol, via ester bonds (which are created when a carboxyl group on a fatty acid reacts with a hydroxy group on an alcohol). For safety and stability, oils of highly purified grade (also called “super refined”) are generally desired, and were used in the tests described below.

The “animal fat” category also includes triglycerides, but the lengths of, and unsaturated bonds in, the three fatty acid chains varies, compared to vegetable oils. Animal fats from sources that are solid at room temperature (such as tallow, lard, etc.) can be processed to render them liquid if desired; other types of animal fats that are inherently liquid at room temperature include various fish oils, oleic acid, etc.

Various synthetic or semi-synthetic oils, such as ethyl oleate, are also known, and can be used if desired.

Triglycerides are typically present in nearly any natural sources of oil or fat, and “medium chain triglycerides” (MCT's, which typically are made from fatty acids that are usually about 8 to 10 carbons in length) have been used extensively in emulsions designed for injection as a source of calories, for patients requiring parenteral nutrition. Numerous monoglycerides and diglycerides which are liquid at room temperature are also known, and may be tested to evaluate their suitability for use as disclosed herein, if desired.

MCT's are generally preferred for use in this invention, because of their long record of safe use in injectable emulsions. However, any other source or type of oil or fat can be evaluated for use as disclosed herein, and specific oils may be identified which are especially useful for preparing taxoid emulsions, using the methods disclosed herein.

Various other types of compounds which have a hydrophobic oily nature and which are liquid at room temperature, are know, and may be suitable for use as an oily carrier as disclosed herein, as can be evaluated using routine experimentation. Some such compounds are of interest in injectable formulations, because they can provide other medical or nutritional benefits; Vitamin E (also known as tocopherol) and various liquid derivatives such as vitamin E acetate (generally known as tocol compounds), and ethyl oleate, are within this class, and can be evaluated for use as disclosed herein if desired, and can be used as an oily carrier if they perform adequately.

Since the oil component is merely a carrier for the active drug, the specific type of oil used is not critical, so long as it is physiologically acceptable and benign, and will provide droplets having a desired size range, and will behave properly (with adequate levels of stability, resistance to collapse and caramelization, etc.) during and after lyophilization, for a period of at least two months (which is generally regarded as a minimally desirable shelf life; it should be noted that various taxoid formulations prepared as disclosed herein appear to be fully stable for at least a year, in tests done to date.

Unless specific factors indicate otherwise, the content of the oil component in the emulsion prior to lyophilization should generally be within a range of about 5 to 50%, by weight. In most cases, an oil content within a range of about 10 to about 30% by weight will be suitable.

B. Surfactants

In this invention, a surfactant is needed to form stable emulsions, and to increase the solubility of drug in the oil phase to a desired concentration. Only a limited number of surfactants are regarded as safe for use in parenteral administration. Natural surfactants include lecithin and various phospholipids, as well as various bile salts and fatty acid salts. Synthetic surfactants which have been approved for injection, at low concentrations, include PLURONIC F68 (also called POLYOXAMER 188), and Tween 80 (also called POLYSORBATE 80).

The surfactant chosen for this type of use may be any suitable surfactant which performs adequately in creating an emulsion as disclosed herein, and which is not aggressively toxic. It should be noted that the emulsions described herein do not need to have shelf lives that extend for weeks or months; the manufactured emulsion, once it has been prepared, normally will be subjected to lyophilization within a matter of minutes or hours, and the reconstituted emulsion, which will be prepared by mixing the cake or powder with water in a hospital or clinic, preferably should also be used within a matter of minutes or hours. Therefore, natural surfactants such as soy lecithin or egg lecithin, which generally will degrade more rapidly than synthetic surfactants once inside the body, are believed and assumed to be preferred over synthetic surfactants, for this use. However, any candidate surfactant which is known to be reasonably nontoxic after injection can be evaluated for use as described herein, using no more than routine experimentation.

Due to their long history of safety, their combined emulsification and solublization properties, and the fact that they tend to be broken down into innocuous substances more rapidly than most synthetic surfactants, soy lecithin and egg lecithin (including hydrogenated versions of these compounds, if desired) are generally preferred for use in this invention.

Any such surfactant may be employed alone, or in combination with other surfactants. For example, soy lecithin and hydrogenated soy lecithin were employed in combination as emulsifying agents. Another example is the combination of soy lecithin and oleic acid.

The content of the surfactant in an emulsion prior to lyophilization should generally be within a range of about 10 to about 100% of the oil weight (rather than the emulsion weight). Ranges of about 30 to 60% of the oil weight are generally preferred, and lecithin concentrations equal to 50% of the oil weight gave good results in various tests done to date.

C. Anti-Adhesion Agents

As indicated above, an anti-adhesion agent is needed to protect the emulsion oil droplets from collapse, and from aggregation, coalescence, caramelization, or other degradation, both during the lyophilization process, and during handling and storage in dry cake or powder form. A suitable anti-adhesion agent might do this in any of several ways, such as: (i) by forming a generally continuous or friable matrix, in which the tiny oil droplets containing the taxoid drug will be suspended; (ii) by forming a clean, fine, dry cake or powder with sufficient bulk to keep most of the oil droplets separated from each other; and/or (iii) by forming dry and non-adhesive coating layers on the surfaces of the oil droplets.

The anti-adhesion agent preferably should also dissolve quickly and readily when water is added, during reconstitution of an emulsion, and in the final emulsion, it should form an innocuous ingredient that will not pose any substantial risk of irritation or toxicity when the emulsion is injected.

Although some researchers have stated a preference for amino acids, or for proteins or hydrolyzed proteins, as anti-adhesion agents, saccharides having a suitable “collapse temperature” are preferred for use herein. Based on tests done to date, it is believed that a saccharide having a collapse temperature of less than about −25° C. is preferred, to prevent collapse of a taxoid material during lyophilization. A saccharide having a collapse temperature higher than about −35° C. is also generally preferred, since conventional lyophilizer machines have difficulty completing a lyophilization process down to a desirably low water level, if they must work with an agent that has a collapse temperature lower than about −35° C. In such a situation, the temperature inside the machine must be reduced to a level significantly below the collapse temeprature; this leads to both greater expense in running machines at extremely low temperatures, and to longer processing times.

With all relevant factors (including processing times and costs) taken into account, sucrose (a disaccharide) is a preferred anti-adhesion agent, and has performed satisfactorily in the formulations described below.

Collapse temperatures are generally the same as or closely related to so-called “glass transition” temperatures, and these indicator numbers are reported, for a variety of compounds, in a number of reference works, such as A. P. MacKenzie, “Basic Principles of Freeze-Drying for Pharmaceuticals,” Bulletin of the Parenteral Drug Association 20(4) (July-August 1966). Because its glass transition temperature reportedly is −32° C., maltose offers a potential candidate for evaluation as an anti-adhesion agent for use with taxoid drugs; however, as noted above, it has not been widely used in injectable formulations, so its presence in an injectable formulation likely would require more extensive clinical trials than sucrose would require, to ensure safety.

Various other potential candidate for use as anti-adhesion agents as disclosed herein may include various mono- or di-saccharides, sugar alcohols, inorganic salts, or hydrophilic polymers. The category of amino acids, proteins, and hydrolyzed protein fragments also can be evaluated, if desired; however, most amino acids tend to be more expensive than saccharides, and proteins can lead to various risks and problems (such as undesired immune or allergic responses) when incorporated into injectable formulations.

If sucrose is used, concentrations in the emulsions prior to lyophilization within the range of about 5 to 30% (measured on a weight/volume basis) are generally preferred. In most cases, a sucrose content within a range of about 10 to about 30% will be suitable.

D. Other Additives

Any other type of additive which serves a desired function (such as an anti-oxidant, a pH buffer, a stabilizer or chelating agent, an anti-microbial agent, a compound to protect against photolytic degradation, a “leaving agent” to help speed up the lyophilization process, hydrogenated phosphatidylcholine or cholesterol to modify the oil phase composition for improved stability, etc.) also can be added to a taxoid emulsion prior to lyophilization, if desired, so long as it does not interfere with the useful traits of the final product. Such agents typically represent about 1% or less, by weight, of the emulsion.

Also, as mentioned above, one or more additional active drugs (which may include a second type of taxoid, if desired) may also be present in a taxoid formulation as disclosed herein. However, a second active drug would not be regarded as a carrier agent, as that term is used herein.

Preferred Formulation Methods

A preferred taxoid formulation according to the principles of the invention is prepared by dissolving paclitaxel in an oil solution containing a suitable oil (such as a medium-chain triglyceride, MCT, mixed with lecithin). If desired, ethanol may be used to facilitate the dissolution of the paclitaxel in the oil and surfactant solution, using the steps shown in FIG. 1. Other fat-soluble additives such as vitamin E may also be dissolved in the oil phase. If an alcohol is used to dissolve the paclitaxel in the oil solution, it should be removed by vacuum evaporation, or evaporation under a stream of nitrogen gas, before emulsification.

Sucrose and other desired water-soluble additives such as sodium EDTA is added to water to form the aqueous phase. The aqueous phase and oil/paclitaxel solution are combined, and the mixture is emulsified with a high-shear homogenizer or probe sonicator.

The resulting primary emulsion is refined by cycling it through a microfluidizer homogenizer, resulting in a stable emulsion having fairly uniform droplet sizes, preferably with average diameters less than a micron, which is suitable for lyophilization. The refined emulsion is filtered through a filter which can remove bacteria and viruses (such as a 0.2 micron filter), and loaded into glass vials. It is lyophilized, in a conventional lyophilization chamber, to form a dry emulsion, using suitable combinations of time, temperature, and vacuum, as indicated in the Examples below. Upon completion of the lyophilization cycle, the vials are sealed under a partial vacuum, with inert gas such as nitrogen in the head space.

Tests completed to date indicate that the dry emulsion is very stable, and is likely to have a shelf life measured in months even when stored at room temperature, without refrigeration.

Shortly before use, which presumably will occur in a hospital or clinic, the dry emulsion is dispersed in sterile water, buffered saline, dextrose solution, or other injectable aqueous liquid, to reform an emulsion or suspension (depending on how much water was added). Adequate dispersion can be accomplished using non-machine mixing, such as shaking the vial or infusion bag by hand. In tests done to date, the reconstituted emulsion has average droplet sizes and size distributions that are very similar to the emulsion prior to lyophilization, as measured by laser light scattering (LLS). The reformed emulsion can be further diluted with water, if desired, and is stable and safe for intravenous injection or infusion.

The claims below refer to certain names taxoid drugs, and to “salts, isomers, derivatives, and analogs thereof which are pharmacologically acceptable and have therapeutic activity in injectable liquid formulations.”

The term “pharmacologically acceptable” embraces those characteristics which make a drug suitable and practical for administration to humans. For example, such compounds must be sufficiently chemically stable under reasonable storage conditions to have an adequate shelf life. They also must be physiologically acceptable when introduced into the body by a suitable route of administration; this implies that, if they cause adverse side effects (such as causing nausea or hair loss, which are common problems among chemotherapeutic agents), then the problems caused by those adverse effects must be outweighed by the therapeutic benefits of the treatment. Clearly, this invention does not relate to new, different, or improved taxoid drugs; instead, it relates to better ways of administering taxoid drugs (either currently known or hereafter discovered) that have been shown by others to be therapeutically useful.

The term “salts” can include alkali metal salts as well as addition salts of free acids or free bases. Examples of acids which may be employed to form pharmaceutically acceptable acid addition salts include inorganic acids such as hydrochloric acid, sulphuric acid and phosphoric acid, and organic acids such as acetic acid, maleic acid, succinic acid, or citric acid. Alkali metal salts or alkaline earth metal salts include, for example, sodium, potassium, calcium, or magnesium salts. All of these salts (or other similar salts) may be prepared by conventional means. The nature of the salt is not critical, provided that it is non-toxic and does not substantially interfere with the desired pharmacological activity.

The term “isomer” as used herein includes conventional isomers (i.e., molecules which have the exact same number of atoms, but in a different arrangement, such as when a certain pendant group is attached to a different carbon atom). It also includes stereoisomers (i.e., molecules in which the four different groups that are attached to a “chiral” carbon atom can be arranged in two possible “mirror image” orientations); as is well known in pharmaceutical chemistry, stereoisomers are receiving substantial attention, and it often turns out that one stereoisomer is safer and/or more potent than its mirror-image stereoisomer.

The term “derivative” is used herein to refer to a molecule that has been derived from a certain known taxoid compound, by carrying out one or more chemical reactions on that taxoid compound.

The term “analog” is used herein in the conventional pharmaceutical sense, to refer to a molecule that structurally resembles a “referent” molecule (a taxine or other taxoid molecule, in this case) but which has been modified in a targeted and controlled manner, to replace a specific substituent of the referent molecule with an alternate substituent (such as, for example, a lower alkyl group, a hydroxy or methoxy group, an amino group, etc.). Synthesis and screening of analogs, to identify slightly modified versions of a known compound which have improved traits (such as higher potency in inhibiting a known enzyme, receptor, or organelle that is overactive in cancer cells, higher selectivity at a targeted cellular surface receptor type coupled with lower activity levels at other receptor types, etc.), are well-known procedures in pharmaceutical chemistry.

### EXAMPLES

### Example 1

### Solubility of Paclitaxel in Oils and Fat Emulsions

These tests were conducted to evaluate the solubility of paclitaxel in various types of oil, with and without the presence of soy lecithin.

To determine the solubility of paclitaxel in oils that were tested without lecithin, paclitaxel was weighed out and mixed with the selected oil. The mixtures were agitated for 24 hours at 25° C., and were then filtered through a 0.2 micron filter. The filtrate was diluted in methanol and analyzed by HPLC for paclitaxel concentration.

To determine solubility of paclitaxel in various oil emulsions containing lecithin, paclitaxel and soy lecithin (Phospholipon 90, Rhone-Poulenc) were weighed out and dissolved in ethanol. The ethanol solution was combined with the selected oil to form a clear yellow solution. The solution was vacuum dried to remove at least 95% of the ethanol content, to obtain the oil phase. A sucrose solution was added, to obtain the final composition of 6 mg/g paclitaxel, 30 mg/g soy lecithin, 100 mg/g oil and 115 mg/g sucrose. The mixture was emulsified using a probe sonifier (Branson Sonifier Model 250). Each emulsion was moderately agitated at 25° C. or at 5° C. for 1 hour and 40 minutes. In some emulsions, visible lumps or aggregates were formed. Emulsion samples with no lumps or aggregates were removed from each sample, and analyzed for paclitaxel concentration.

Table 1 shows the paclitaxel solubility in these oil emulsions; references in Table 1 to emulsions indicate that soy lecithin was also present in those mixtures.

TABLE I
SOLUBILITY OF PACLITAXEL IN OILS AND EMULSIONS
CONTAINING 30 MG/G SOY LECITHIN, 100 MG/G OIL AND
115 MG/G SUCROSE IN WATER

| | Solubility at 5° C. | Solubility at 25° C. |
|---|---|---|
| Oil | (mg/g of oil) | (mg/g of oil) |
| Sesame oil ^{a} | Not determined | 5 |
| MCT oil ^{b} | Not determined | 40 |
| Corn oil emulsion ^{c} | 15 | 10 |
| Cottonseed oil emulsion ^{a} | 20 | 9 |
| Safflower oil emulsion ^{a} | 12 | 9 |
| Sesame oil emulsion | 30 | 20 |
| Soybean oil emulsion ^{a} | 10 | 5 |
| Ethyl oleate emulsion ^{d} | 6 | 5 |
| Vitamin E emulsion ^{e} | 0 | 0 |
| MCT emulsion | 22 | 14 |

^{a} Super refined oil, Croda Inc.
^{b} Miglyol 812N, Condea Chemie GmbH
^{c} NF grade, Spectrum Chemical Mfg Corp.
^{d} NF grade, Spectrum Chemical Mfg Corp.
^{e} 97%, Aldrich Chemical

These results indicate that paclitaxel is more soluble in emulsions containing sesame or MCT oil, than in other oils that have been evaluated to date. Paclitaxel solubility in sesame oil can be enhanced significantly by soy lecithin in an emulsion form, thereby achieving a reasonably high concentration (2 to 3 mg/g) in the emulsion.

### Example 2

### Stability of Paclitaxel in Oils

These tests were conducted to evaluate oils that are compatible with paclitaxel. Soy lecithin (PHOSPHOLIPON 90, sold by Rhone-Poulenc) and paclitaxel were weighed out and dissolved in ethanol. The ethanol solution was mixed with the selected oil. The oil solution was subjected to a vacuum dry to remove at least 95% of the ethanol content. The resulting oil solutions were sealed in glass vials and stored at 40° C. or at 60° C. Aliquots were removed at day 0, day 3 and day 7 for HPLC analysis for paclitaxel concentration and purity.

The results, in Table 2, indicate that MIGLYOL 812N and sesame oils, in the presence of soy lecithin, were the most compatible oils among the oils tested. The presence of soy lecithin significantly improved the stability of paclitaxel in sesame oil.

TABLE 2
STABILITY OF PACLITAXEL IN
SOLUTIONS CONTAINING OIL AND SOY LECITHIN

Purity (% paclitaxel peak
area over total peak area)

| | | Stored at 60° C. |
|---|---|---|
| Composition | Day 0 | for 36 days |
| Corn oil | 98.7 | 71.5 |
| Cottonseed | 98.8 | 94.2 |
| Safflower | 98.0 | 94.7 |
| Sesame | 99.0 | 96.3 |
| Soybean | 99.4 | 94.2 |
| Miglyol 812N | 99.3 | 96.0 |
| Ethyl oleate | 97.0 | 91.7 |
| Vitamin E | 96.3 | 34.2 |
| Sesame oil + oleic acid | 98.5 | 94.8 |
| Sesame oil + vit E | 99.0 | 88.0 |
| Sesame oil + water | 99.1 | 95.9 |
| Sesame oil without soy lecithin | 99.2 | 75.1 |

### Example 3

### Preparation of Lyophilized Emulsions Using MCT, Soy Lecithin and Dextrose

Soy lecithin (9.35 g Phospholipon 90) was weighed out and mixed with 7.37 g MCT (Migyol 812N) and 0.99 g dehydrated ethanol. The mixture was sonicated at 50° C. for about 1.5 hours to form a clear yellow solution. The solution was diluted separately with 5% dextrose solution for injection USP (D5W) by 3, 10 and 30 folds (Table 3) to a final weight of approximately 1 g. Each diluted mixture was filled into a plastic vial and subjected to a vigorous agitation using a mini-beadbeater (Biospec Products) for 5 minutes. The resulting emulsions were subjected to 8 minutes of vacuum to remove air bubbles. The emulsions were diluted with D5W for measurement of droplet size using a laser light scattering spectrometer (LLS).

One milliliter of each emulsion was filled into a 5 ml glass vial. The vials were placed on a shelf of a freeze-dryer (Dura-Stop MP by FTS Systems). The shelf temperature was brought down to −40° C. at 1° C./min rate and held at −40° C. for 30 minutes. The frozen emulsions were then subjected to a vacuum drying at 50 millitorr while the shelf temperature was held at −20° C. for 12 hours. The shelf temperature was then raised to 20° C. and held at 20° C. for 2.5 hours to complete the freeze-drying cycle.

The lyophilizates were reconstituted with 1 ml deionized water to reform the emulsions. The reformed emulsions were further diluted with D5W for droplet size measurement using a laser light scattering spectrometer (Zetasizer 1000 HSA, Malvern Instruments).

The lyophilizate appearance, dispersion rate and droplet size of the emulsions before lyophilization and after reconstitution are listed in Table 3.

TABLE 3
LYOPHILIZED EMULSIONS CONTAINING MCT,
SOY LECITHIN AND DEXTROSE

| | | Average Droplet | |
|---|---|---|---|
| | | Size (nm) | |
| | | Before | After |
| Pre-freeze-drying | | freeze- | reconsti- |
| emulsion Composition | Appearance | drying | tution |
| 17% w/w PL90 | Yellow cake with | 203 ± 0.9 | 196 ± 0.4 |
| 13.4% w/w Miglyol | shrinkage. Dis- | | |
| 812N | persed slowly to | | |
| 1.8% Ethanol | form thick paste | | |
| 3.4% w/w dextrose | | | |
| 64.4% w/w water | | | |
| 5.2% w/w PL90 | Off-white cake with | 206 ± 1.7 | 196.4 ± 0.4 |
| 4.1% w/w Miglyol | uniform appearance | | |
| 812N | and dispersed | | |
| 0.6% Ethanol | rapidly in water to | | |
| 4.5% w/w dextrose | form a uniform | | |
| 85.6% w/w water | emulsion | | |
| 1.8% w/w PL90 | Off-white cake with | 232 ± 2.6 | 196.7 ± 1.3 |
| 1.4% w/w Miglyol | uniform appearance | | |
| 812N | and dispersed | | |
| 0.2% Ethanol | rapidly in water to | | |
| 4.8% w/w dextrose | form a uniform | | |
| 91.8% w/w water | emulsion | | |

These results indicate it is possible to freeze-dry a submicron emulsion containing soy lecithin, MCT and disaccharide to form a dry emulsion, which can be dispersed in water to reform the emulsion without any increase in average droplet size.

### Example 4

### Preparation of Lyophilized Emulsion Using MCT, Soy Lecithin and Various Anti-Adhesion Agents

MCT or soybean oil, soy lecithin and ethanol were weighed and mixed to form a clear solution. The solution was dried under vacuum to remove ethanol to more than 95% to form the oil phase. The selected freeze-drying aid was weighed out and dissolved in water to form the aqueous phase. A primary emulsion (30 ml) was prepared by mixing the oil and aqueous phases using a high shear emulsifier (Model L4RT, Silverson Machine Ltd.) at 11,000 RPM for 30 seconds. The primary emulsion was then homogenized to form the final emulsion at room temperature by passing through a Microfluidizer homogenizer (Model 110S, Microfluidics Corp.) equipped with an interaction chamber (Model F20Y, 75 A) for 10 cycles at 18,000 psi operation pressure. Average droplet size of the final emulsion was determined using LLS.

One milliliter of each emulsion was filled into 5 ml glass vials and lyophilized using the following conditions:

The shelf was chilled at 1° C./min to −40° C., then held at −40° C. for 60 min. The shelf was then held at −40° C. with vacuum at 100 mTorr for 10 min, then at −10° C. with vacuum at 100 mTorr for 2880 min. The shelf was then raised to 20° C. with vacuum at 100 mTorr over 240 min. The dry emulsion lyophilizates were dispersed in water and measured for average droplet size using LLS.

The emulsion compositions and droplet sizes, both before lyophilization and after reconstitution, are listed in Table 4. These results indicate that disaccharides (trehalose and sucrose) are the preferred freeze-drying aids of the agents tested, and sucrose provided the best results seen to date. These results also indicated that MCT emulsion appears to be better suited for lyophilization than the soybean oil emulsion.

### Example 5

### Preparation of Lyophilized Emulsions Containing Paclitaxel

Paclitaxel, MCT or sesame oil, soy lecithin and ethanol were weighed and mixed to form a clear oil solution. The solution was dried under vacuum to remove ethanol to more than 95% to form the oil phase. The selected freeze-drying aid was weighed out and dissolved in water for form the aqueous phase.

A primary emulsion (14 ml) was prepared by mixing the oil and aqueous phases using a probe sonifier (Branson Sonifier model 250) using settings at 50% duty cycle and 50 output for

TABLE 4
Lyophilized emulsions with various oils and anti-adhesion agents

| | Average Droplet | |
|---|---|---|
| | Size (nm) | |
| | Before | After |
| | freeze- | reconsti- |
| Pre-freeze-drying emulsion Composition (w/w) | drying | tution |
| 10% MCT, 13% soy lecithin, 1.3% hydrogenated | 150 | 155 |
| soy lecithin, 10% trehalose and water to QS | | |
| 10% MCT, 3% soy lecithin, 1.3% hydrogenated | 179 | 192 |
| soy lecithin, 5% trehalose and water to QS | | |
| 5% MCT, 13% soy lecithin, 1.3% hydrogenated | 158 | 261 |
| soy lecithin, 5% trehalose and water to QS | | |
| 10% Soybean oil, 13% soy lecithin, 1.3% hydro- | 174 | 300 |
| genated soy lecithin, 5% trehalose and water to QS | | |
| 10% MCT, 13% soy lecithin, 1.3% hydrogenated | 143 | 754 |
| soy lecithin, 5% dextran 40K and water to QS | | |
| 10% MCT, 13% soy lecithin, 1.3% hydrogenated | 157 | 752 |
| soy lecithin, 5% glycine and water to QS | | |
| 10% MCT, 13% soy lecithin, 1.3% hydrogenated | 152 | 1324 |
| soy lecithin, 5% sodium chloride and water to QS | | |
| 10% MCT, 13% soy lecithin, 1.3% hydrogenated | 161 | 1128 |
| soy lecithin, 5% alpha cyclodextrin and water to QS | | |
| 10% MCT, 13% soy lecithin, 1.3% hydrogenated | 177 | 604 |
| soy lecithin, 5% mannitol and water to QS | | |
| 10% MCT, 13% soy lecithin, 1.3% hydrogenated | 173 | 443 |
| soy lecithin, 2.5% mannitol, 2.5% dextran 40K and | | |
| water to QS | | |
| 10% MCT, 3% soy lecithin, 20% hydrogenated | 379 | 844 |
| cyclodextrin and water to QS | | |
| 10% MCT, 3% soy lecithin, 20% sucrose and water | 166 | 144 |
| to QS | | |

4 minutes. The primary emulsion was then homogenized to form the final emulsion at room temperature by passing through a Microfluidizer homogenizer (Model 110S, Microfluidics Corp.) equipped with an interaction chamber (Model F20Y, 75μ) for 10 cycles at 18,000 psi operation pressure. The emulsion was filtered through a 0.2 micron nylon filter. Average droplet size of the filtered emulsion was determined using LLS.

An aliquot (0.2 ml) of each emulsion was filled into 2 ml glass vials and lyophilized using the following conditions. The shelf was chilled at 1° C./min to −40° C., and held at −40° C. for 30 min. The vacuum was then reduced to 50 mTorr over 30 min, and held at −35° C. and 50 mTorr for 2160 min. The shelf was then raised to 30° C. with vacuum at 50 mTorr over 480 min.

The dry emulsion lyophilizates were dispersed in water and measured for average droplet size using LLS. The emulsion composition and droplet size before lyophilization and after reconstitution are listed in Table 5.

TABLE 5
DROPLET DIAMETERS FOR DRY PACLITAXEL EMULSIONS

| | Average Droplet Size (nm) | |
|---|---|---|
| Pre-freeze-drying emulsion | Before | After |
| Composition (w/w) | freeze-drying | reconstitution |
| 2.5 mg/g paclitaxel, 15% MCT, 7.5% soy | 201 ± 2 | 199 ± 2 |
| lecithin, 15% sucrose and water to QS | | |
| 0.8 mg/g paclitaxel, 15% sesame oil, 7.5% | 116 ± 2 | 477 ± 9 |
| soy lecithin, 15% sucrose and water to QS | | |

These results indicate that dry emulsions of paclitaxel can be prepared by lyophilization in a composition containing 0.8±2.5 mg/g paclitaxel, 15% oil, 7.5% soy lecithin and 15% water. MCT appeared to be preferred over sesame oil since there was no droplet size increase in the MCT emulsion upon lyophilization.

### Example 6

### Paclitaxel Stability Study #1: MCT vs. Sesame Oil

Sample of the freeze-dried emulsions containing paclitaxel as described in example 5 were stored at −20° C., 2-8° C., 25° C. and 40° C. for stability evaluation. At each time point including the time 0, a vial was removed from each stability temperature chamber and reconstituted with 0.2 ml deionized water to reform the emulsion. The reformed emulsion was diluted with water for LLS measurement for droplet size and with methanol for HPLC analysis of paclitaxel concentration and purity.

Along with the dry emulsion samples, the liquid emulsion samples (pre-lyophilization) were also included in this study. The liquid emulsions were stored at −20° C.

Table 6 lists the concentration recovery of paclitaxel and sample purity as measured by percente of the peak area of paclitaxel. These results indicate that droplets of MCT emulsions are more stable than the sesame oil emulsions during the freeze-drying process and freeze-thaw treatment. These results also indicated that paclitaxel is chemically stable for at least 28 days in the dry emulsions of MCT or sesame oil at a storage temperature of 25° C. or below. The variation observed in concentration recovery was believed the result of assay variability. The recovery of paclitaxel was supported by the purity measurements.

### Example 7

### Paclitaxel Emulsion Stability Study #2: Effect of pH and Dry Emulsions vs. Liquid Emulsions

These tests were conducted to select optimal pH's for a dry paclitaxel emulsion. Paclitaxel, MCT, soy lecithin and ethanol were weighed and mixed to form a clear solution. The solution was dried with a stream of nitrogen gas to remove ethanol to more than 95% to form the oil phase. Sucrose was weighed out and dissolved in water for form the aqueous phase.

A primary emulsion (14 ml) was prepared by mixing the oil

TABLE 6
Stability of freeze-dried and liquid emulsions containing paclitaxel

| | Emulsion | Temp | | | | | |
|---|---|---|---|---|---|---|---|
| Pre-freeze-drying emulsion | Form | ° C. | | Initial | Day 7 | Day 14 | Day 28 |
| | | | | Concentration Recovery (% of the Initial) | | | |
| 2.5 mg/g paclitaxel | Dry | −20° | C. | 100.0 | 110.9 | 96.8 | 99.8 |
| | Dry | 2-8° | C. | 100.0 | 92.0 | 98.0 | 98.2 |
| | Dry | 25° | C. | 100.0 | 94.8 | 96.5 | 98.0 |
| | Dry | 40° | C. | 100.0 | 94.2 | 94.4 | 95.9 |
| | Liquid | −20° | C. | 100.0 | 98.2 | 95.6 | 97.3 |
| 0.8 mg/g paclitaxel | Dry | −20° | C. | 100.0 | 102.4 | 99.3 | 105.3 |
| | Dry | 2-8° | C. | 100.0 | 104.1 | 102.5 | 106.9 |
| | Dry | 25° | C. | 100.0 | 99.3 | 102.7 | 105.4 |
| | Dry | 40° | C. | 100.0 | 102.6 | 100.3 | 105.7 |
| | Liquid | −20° | C. | 100.0 | 97.1 | 100.3 | 99.9 |
| | | | | Purity (% of paclitaxel peak area over total) | | | |
| 2.5 mg/g paclitaxel | Dry | −20° | C. | 99.4 | 99.4 | 99.3 | 99.4 |
| | Dry | 2-8° | C. | 99.4 | 99.6 | 99.5 | 99.6 |
| | Dry | 25° | C. | 99.4 | 99.7 | 99.5 | 98.9 |
| | Dry | 40° | C. | 99.3 | 99.5 | 99.2 | 98.5 |
| | Liquid | −20° | C. | 99.3 | 99.5 | 99.3 | 99.4 |
| 0.8 mg/g paclitaxel | Dry | −20° | C. | 99.3 | 99.6 | 99.3 | 99.7 |
| | Dry | 2-8° | C. | 99.3 | 99.4 | 99.3 | 99.3 |
| | Dry | 25° | C. | 99.3 | 99.3 | 99.6 | 99.4 |
| | Dry | 40° | C. | 99.3 | 99.3 | 99.0 | 99.3 |
| | Liquid | −20° | C. | 99.3 | 99.4 | 99.2 | 99.3 |

Average Droplet Size (nm)

| | | | | | | | |
|---|---|---|---|---|---|---|---|
| 2.5 mg/g paclitaxel | Dry | −20° | C. | 201 | 207 | 208 | 217 |
| | Dry | 2-8° | C. | 201 | 202 | 208 | 224 |
| | Dry | 25° | C. | 201 | 219 | 211 | 213 |
| | Dry | 40° | C. | 201 | 263 | 282 | 214 |
| | Liquid | −20° | C. | 199 | 192 | 197 | 204 |
| 0.8 mg/g paclitaxel | Dry | −20° | C. | 477 | 537 | 533 | 519 |
| | Dry | 2-8° | C. | 477 | 508 | 526 | 556 |
| | Dry | 25° | C. | 477 | 477 | 476 | 692 |
| | Dry | 40° | C. | 477 | 391 | 372 | 599 |
| | Liquid | −20° | C. | 116 | 261 | 273 | 278 |

and aqueous phases using a probe sonifier (Branson Sonifier model 250) using setting at 50% duty cycle and 50 output for 4 minutes. The primary emulsion was then homogenized to form the final emulsion at room temperature by passing through Microfluidizer homogenizer (Model 110S, Microfluidics Corp.) equipped with an interaction chamber (Model F20Y, 75μ) for 10 cycles at 18,000 psi operation pressure. The emulsion was filtered through a 0.2 micron nylon filter. The fine emulsion was divided into 5 portions. The pH of each portion was adjusted to pH 4.0, 5.6, 6.6, 7.8 or 8.9 using 0.2 N HCl or 0.2 N NaOH solution. Average droplet size of each pH adjusted emulsion was determined using LLS. The emulsion compositions are listed in Table 7.

TABLE 7
Emulsion compositions
Pre-freeze-drying emulsion Composition (w/w)
2.5 mg/g paclitaxel, 15% MCT, 7.5% soy lecithin, 15% sucrose and
water to QS, pH 4.0
2.5 mg/g paclitaxel, 15% MCT, 7.5% soy lecithin, 15% sucrose and
water to QS, pH 5.6
2.5 mg/g paclitaxel, 15% MCT, 7.5% soy lecithin, 15% sucrose and
water to QS, pH 6.6
2.5 mg/g paclitaxel, 15% MCT, 7.5% soy lecithin, 15% sucrose and
water to QS, pH 7.8

An aliquot (0.2 ml) of each emulsion was filled into 2 ml glass vials and lyophilized. The shelf was chilled at 1° C./min to −40° C., and held at −40° C. for 30 min. Vacuum was reduced to 50 mTorr over 30 min, and held at −35° C. and 50 mTorr for 2160 min. The shelf was warmed to 30° C., with vacuum at 50 mTorr, over 480 min.

The stability evaluation included both dry and liquid emulsions (pre-lyophilization) at each pH. These emulsions were stored sealed in glass vials at 40° C. At each time point, the contents were either diluted with water for the liquid emulsion or reconstituted with water for the dry emulsion for droplet size measurement by LLS and paclitaxel concentration and purity by HPLC.

Droplet size analysis indicated that: (1) the dry emulsions appeared to be most stable at pH 6.6; (2) the droplet stability of the dry and liquid emulsions were comparable at 40° C.; and, (3) paclitaxel in dry emulsions was more stable than in the liquid emulsions at pH 4.0 to 8.9.

These results further suggested that highly useful paclitaxel emulsions can be prepared by lyophilizing a liquid submicron emulsion containing 2.5 mg/g paclitaxel, 15% w/w MCT, 7.5% w/w soy lecithin and 15% w/w sucrose at pH 6.6.

Thus, there has been shown and described a new and useful means for creating stable and benign lyophilized preparations, which can be reconstituted into taxoid emulsions by mixing with water. Although this invention has been exemplified for purposes of illustration and description by reference to certain specific embodiments, it will be apparent to those skilled in the art that various modifications, alterations, and equivalents of the illustrated examples are possible. Any such changes which derive directly from the teachings herein, and which do not depart from the spirit and scope of the invention, are deemed to be covered by this invention.

1. A composition of matter, comprising a porous lyophilized formulation containing at least one taxoid drug, wherein the formulation can be readily reconstituted into an injectable suspension or emulsion by mixing with water using mild agitation that does not require machine processing, and wherein:
a. the taxoid drug is hydrophobic and insoluble in water, and is contained in oily droplets;
b. the oily droplets contain or are coated by a benign surfactant compound that can be readily tolerated by essentially all patients;
c. the formulation also contains an anti-adhesion agent which (i) prevents agglomeration of the particles while in lyophilized form, (ii) is readily soluble in water, and (iii) has been shown to prevent collapse of the formulation during lyophilization,
and wherein the formulation remains stable and does not collapse or deteriorate significantly when stored at room temperature for two months in a sealed vial, and wherein the formulation also is characterized by absence of any carrier ingredients that cause hypersensitivity or pain at injection sites.

2. The composition of claim 1, wherein the taxoid drug comprises a taxine drug.

3. The composition of claim 1, wherein the taxoid drug is selected from the group consisting of paclitaxel; taxotere; taxane; spicatin; yunnanxol; taxane-2,13-dione,5β,9β,10β-trihydroxy-cyclic-9,10-acetal with acetone or acetate; taxane-2,13-dione,5β,9β,10β-trihydroxy-cyclic-9,10-acetal with acetone or acetate; taxane-2β,5β,9β,10β-tetrol-cyclic-9,10-acetal with acetone or acetate; N-debenzoyltaxol A; cephalomannine; cephalomannine-7-xyloside; 7-epi-10-deacetyl-cephalomannine; 10-deacetyl-cephalomannine; baccatin; baccatin diacetate; baccatin I through VI; 7-epi-baccatin III; baccatin A; 7-(4-azido-benzoyl)-baccatin III; O-acetylbaccatin IV; 7-(triethylsilyl)-baccatin III; 7,10-di-O-[(2,2,2-trichloroethoxy)-carbonyl]-baccatin III; 13-(2′,3′-dihydroxy-3′-phenylpropionyl)-baccatin III; baccatin III 13-O-acetate; taxol B; epitaxol; 10-deacetyl-7-epitaxol; 10-deacetyltaxol; 10-deacetyltaxol B or C; 7-xylosyl-10-deacetyltaxol; and 10-deacetyltaxol-7-xyloside; and salts, isomers, derivatives, and analogs thereof which are pharmacologically acceptable and have therapeutic activity in injectable liquid formulations.

4. The composition of claim 1, wherein the surfactant compound is a lecithin compound isolated from a naturally occurring source.

5. The composition of claim 1, wherein the oily droplets comprise a medium-chain triglyceride compound.

6. The composition of claim 1, wherein the anti-adhesion agent has a collapse temperature in a range of about −25° C. to about −30° C.

7. The composition of claim 6, wherein the anti-adhesion agent comprises a saccharide.

8. The composition of claim 7, wherein the anti-adhesion agent comprises sucrose.

9. An article of manufacture, comprising a sealed vial and a sterile porous lyophilized formulation containing a taxoid drug contained within the vial, wherein the formulation can be readily reconstituted into an injectable suspension or emulsion by mixing with water using mild agitation that does not require machine processing, and wherein:
d. the taxoid drug is hydrophobic and insoluble in water, and is contained in oily droplets;
e. the oily droplets contain or are coated by a benign surfactant compound that can be readily tolerated by essentially all patients;
f. the formulation also contains an anti-adhesion agent which (i) prevents agglomeration of the particles while in lyophilized form, (ii) is readily soluble in water, and (iii) has been shown to prevent collapse of the formulation during lyophilization,
and wherein the formulation remains stable and does not collapse or deteriorate significantly when stored at room temperature for two months in a sealed vial, and wherein the formulation also is characterized by absence of any carrier ingredients that cause hypersensitivity or pain at injection sites.

10. The article of claim 9, wherein the taxoid drug comprises a taxine drug.

11. The article of claim 9, wherein the taxoid drug is selected from the group consisting of paclitaxel; taxotere; taxane; spicatin; yunnanxol; taxane-2,13-dione,5β,9β,10β-trihydroxy-cyclic-9,10-acetal with acetone or acetate; taxane-2,13-dione,5β,9β,10β-trihydroxy-cyclic-9,10-acetal with acetone or acetate; taxane-2β,5β,9β,10β-tetrol-cyclic-9,10-acetal with acetone or acetate; N-debenzoyltaxol A; cephalomannine; cephalomannine-7-xyloside; 7-epi-10-deacetyl-cephalomannine; 10-deacetyl-cephalomannine; baccatin; baccatin diacetate; baccatin I through VI; 7-epi-baccatin III; baccatin A; 7-(4-azido-benzoyl)-baccatin III; O-acetylbaccatin IV; 7-(triethylsilyl)-baccatin III; 7,10-di-O-[(2,2,2-trichloroethoxy)-carbonyl]-baccatin III; 13-(2′,3′-dihydroxy-3′-phenylpropionyl)-baccatin III; baccatin III 13-O-acetate; taxol B; epitaxol; 10-deacetyl-7-epitaxol; 10-deacetyltaxol; 10-deacetyltaxol B or C; 7-xylosyl-10-deacetyltaxol; and 10-deacetyltaxol-7-xyloside; and salts, isomers, derivatives, and analogs thereof which are pharmacologically acceptable and have therapeutic activity in injectable liquid formulations.

12. The article of claim 9, wherein the surfactant compound is a lecithin compound isolated from a naturally occurring source.

13. The article of claim 9, wherein the oily droplets comprise a medium-chain triglyceride compound.

14. The composition of claim 9, wherein the anti-adhesion agent has a collapse temperature in a range of about −25° C. to about −35° C.

15. The composition of claim 9, wherein the anti-adhesion agent comprises a saccharide.

16. The composition of claim 9, wherein the anti-adhesion agent comprises sucrose.

17. A method of manufacturing a porous lyophilized formulation containing a taxoid drug, comprising the following steps:
a. preparing a first solution containing the taxoid drug in an oily liquid carrier substance which contains a surfactant, and a second solution containing water and an anti-adhesion agent which (i) prevents agglomeration of oily droplets containing the taxoid drug in a lyophilized emulsion, (ii) is readily soluble in water, and (iii) has been shown to prevent collapse of an emulsion which contains the taxoid drug, during and after lyophilization of the emulsion;
b. mixing the first and second solutions at a volume ratio which can be used to create an oil-in-water emulsion;
c. treating the mixed first and second solutions in a manner which creates an emulsion having an average droplet size of less than about 2 microns;
d. loading the emulsion into a plurality of lyophilization vials;
e. subjecting the emulsion in the vials to a freezing temperature which is below the collapse temperature of the anti-adhesion agent, to create a stable frozen emulsion;
f. subjecting the frozen emulsion to vacuum conditions for a sufficient period of time to create a stable and non-collapsed lyophilized cake or powder within the vials; and,
g. sealing the vials which contain the stable and non-collapsed lyophilized cake or powder.

18. The method of claim 17, wherein the emulsion is passed through a sterilization filter as it is being loaded into lyophilization vials.

19. An article of manufacture, comprising a sealed vial and a sterile porous lyophilized formulation, prepared by the method of claim 17, contained within the vial.
